(19) United States
(12) Patent Application Publication
(10) Pub. No.: US 2019/0083377 A1
(43) Pub. Date: Mar. 21, 2019

(54) FOOD COMPOSITION AND COSMETIC COMPOSITION FOR IMPROVING SKIN BEAUTY, COMPRISING SUGAR SOLUTION WITH HIGH GALACTOOLIGOSACCHARIDE CONTENT

(71) Applicant: NEO CREMAR CO., LTD., Seoul (KR)

(72) Inventors: Jae Hwan KIM, Seoul (KR); Hyung-Joo SUH, Seoul (KR); Jung-Cheul SHIN, Seoul (KR); Na-Ri KIM, Seoul (KR)

(21) Appl. No.: 15/850,715

(22) Filed: Dec. 21, 2017

(30) Foreign Application Priority Data
Sep. 21, 2017 (KR) 10-2017-0121958
Dec. 13, 2017 (KR) 10-2017-0172260

(51) Int. Cl. A61K 8/60; A61K 8/73; C07H 3/06; A61Q 19/00; A23L 27/30

(52) U.S. Cl. CPC A61K 8/60; A61K 8/73; C07H 3/06; A61Q 19/007; A23V 2250/612; A23V 2002/00; A23V 2200/318; A23V 2250/608; A23V 2250/61; A23L 27/33

(57) ABSTRACT

The present invention relates to a food composition and a cosmetic composition for improving skin beauty, which include galactooligosaccharide and have an ability to increase a skin water holding capacity, an ability to suppress transepidermal water loss, an ability to alleviate wrinkles, and an ability to alleviate erythema.

### CROSS-REFERENCE TO RELATED APPLICATION

This application claims priority to and the benefit of Korean Patent Application No. 10-2017-0121958, filed on Sep. 21, 2017, and Korean Patent Application No. 10-2017-0172260, filed on Dec. 13, 2017, the disclosures of which are incorporated herein by reference in their entirety.

### BACKGROUND

### 1. Field of the Invention

The present invention relates to a food composition and a cosmetic composition, which include galactooligosaccharide and improve skin beauty. The term “improving skin beauty” means an increase in skin water holding capacity, a suppression of transepidermal water loss, an alleviation of skin wrinkles, and an alleviation of skin erythema.

### 2. Discussion of Related Art

Skin in the human body is a portion which is in contact with the external environment and serves to protect the inside of the human body from various factors in the external environment. Traditionally, cosmetics have been used in order to improve and preserve skin conditions. However, as inner beauty has become popular recently, beauty foods for achieving both health and skin beauty have drawn attention. For example, Korean Patent No. 10-1349246 relates to a health functional food composition for improving skin beauty, containing ginseng fruit extracts, and Korean Patent No. 10-1449282 relates to a food composition and a cosmetic composition for alleviating skin wrinkles and moisturizing the skin, containing barley fermented by Pichia jadinii and Aureobasidium pullulans bacteria.

The aging of the skin may be largely divided into intrinsic aging and extrinsic aging (photo aging). For the intrinsic aging, as a person ages, functions of fibroblasts are attenuated and the number of cells are decreased, and as a result, the synthesis amount of extracellular matrix protein fibers such as collagen, elastin, and fibrillin is decreased and the structure becomes loose, thereby reducing elasticity, and moisture in skin cells is lost, and as a result, the structure of the stratum corneum is changed and the function of collagenase is increased, so that the crosslinked form of collagen is increased, thereby resulting in a decrease in moisturization. For the extrinsic aging (photo aging), active oxygen species is generated by the stimulus of UV rays, and as a result, production of cytokines is promoted, thereby activating a signal transduction system. In addition, the stimulus of UV rays suppresses TGF-beta and TGF-alfa by activating the activator protein (AP-1), thereby reducing the synthesis of collagen I and collagen II which are main components of the dermis. Activation of AP-1 and NF-kB activates matrix metalloproteinases (MMPs), thereby promoting the degradation of connective tissue in the dermis. In the mechanism for the generation of skin wrinkles, the skin exposure caused by aging, UV rays, and the like activates the mitogen-activated protein kinase (MARK), and the factor which most significantly affects the MARK is AP-1. AP-1 is activated by external environmental stimuli such as UV rays. Furthermore, AP-1 adjusts the expression of a variety of genes associated with cells and differentiation, and strongly adjusts the expression of MMPs, and the MMPs are enzymes that degrade extracellular matrix proteins, and serve to degrade collagen through the signal transduction caused by the stimulus of UV rays. When the expression of MMPs is suppressed, degradation of collagen in the skin is inhibited, and as a result, it is possible to have effects of increasing a skin water holding capacity, suppressing transepidermal water loss, alleviating skin wrinkles, and the like. Thus, the present inventors have studied food and a cosmetic containing galactooligosaccharide for improving skin beauty, and have found that there is an effect of improving skin beauty by components other than a general galactooligosaccharide, thereby completing the present invention.

### SUMMARY OF THE INVENTION

The present invention is directed to providing a food composition and a cosmetic composition, containing galactooligosaccharide, which has a skin moisturizing ability, a skin wrinkle alleviating ability, and an erythema alleviating ability.

According to an aspect of the present invention, there are provided a food composition and a cosmetic composition for improving skin beauty, including a galactooligosaccharide in which galactosyllactose and tetra or higher saccharides of galactooligosaccharide are enhanced as active ingredients.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 illustrates structures of galactobiose, allolactose, and galactosyllactose in galactooligosaccharide;

FIG. 2 is a schematic view illustrating a method for preparing a sugar solution in Example 1;

FIG. 3 illustrates the MMP-2 inhibition ability of sugar solutions in Example 1 and Comparative Examples 1 to 4;

FIG. 4 illustrates the MMP-9 inhibition ability of sugar solutions in Example 1 and Comparative Examples 1 to 4;

FIG. 5 illustrates a change in skin water holding capacity during the ingestion of the sugar solution in Example 1;

FIG. 6 illustrates a change in transepidermal water loss during the ingestion of the sugar solution in Example 1;

FIG. 7 illustrates changes in skin wrinkle area and wrinkle depth during a treatment with the sugar solution in Example 1; and

FIG. 8 illustrates a change in skin condition during a treatment with the sugar solution in Example 1.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

The present invention relates to a food composition including a sugar solution including galatooligosaccharide and having an ability to increase a skin water holding capacity.

The present invention also relates to a cosmetic composition including a sugar solution including galatooligosaccharide and having an ability to increase a skin water holding capacity.

The present invention relates to a method for increasing a skin water holding capacity, the method including: administering a composition including a sugar solution including galactooligosaccharide to a subject.

Hereinafter, the present invention will be described in detail.

Sugar Solution with High Galactooligosaccharide Content

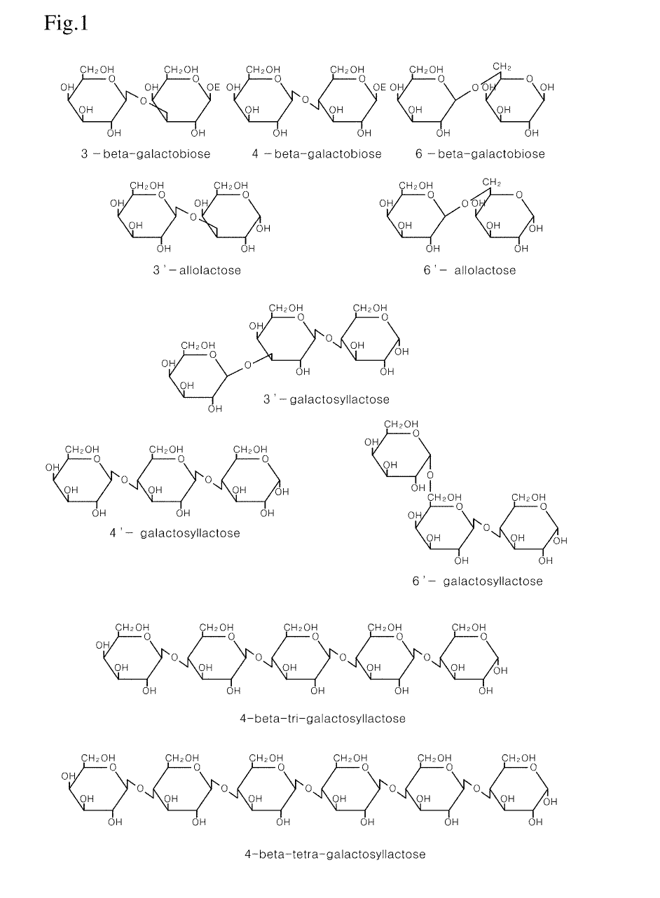

The present invention relates to a food composition and a cosmetic composition, which include a sugar solution with a high galactooligosaccharide content, and has skin moisturizing ability and transepidermal water loss suppressing ability and alleviate skin wrinkles and erythema. The sugar solution is a mixture of various sugars including galactooligosaccharide. The galactooligosaccharide is a mixture of a plurality of sugars. The galactooligosaccharide includes monosaccharides of glucose and galactose, galactobiose, lactose, and allolactose, galactosyllactose which is a trisaccharide, and other tetra or higher saccharides of galactooligosaccharide. The galactobiose may include 4-beta-galactobiose and 6-beta-galactobiose. The allolactose may include 6-galactosyl-glucose. The beta galactosyllactose may include 3′-galactosyllactose, 4′-galactosyllactose, 6′-galactosyllactose, 4-beta-di-galactosyllactose, 4-beta-tri-galactosyllactose, 4-beta-tetra-galactosyllactose, and the like (FIG. 1).

Preferably, the sugar solution of the present invention includes the disaccharide galactobiose and a tri or higher saccharide galactooligosaccharide, which includes allolactose and galactosyllactose. Among sugars in galactooligosaccharide, galactobiose and allolactose may be included in an amount of 15% to 22% based on anhydrides of the sugar solution. The trisaccharide galactosyllactose may be included in an amount of 35% to 55% based on the anhydrides of the sugar solution. Tetra or higher saccharides of galactooligosaccharide may be included in an amount of 10% to 20% based on the anhydrides of the sugar solution. Further, in the case of glucose and galactose among sugars in a galactooligosaccharide sugar solution of the present invention, the sum thereof may be included in an amount of 3% to 8% based on the anhydrides of the sugar solution. Lactose may be included in an amount of 5% to 18% based on the anhydrides of the sugar solution. At this time, among sugars in the sugar solution, the amount of galactosyllactose is larger than the combined amount of galactobiose and allolactose.

The sugar solution of the present invention suppresses transepidermal water loss and suppresses wrinkles and erythema.

Preparation of Sugar Solution with High Galactooligosaccharide Content

After lactose (milk sugar) is introduced into a reaction tank, hot water is added thereto, and the concentration of the milk sugar solution is adjusted to about 40 to 45% by operating a stirrer. When the concentration of the milk sugar solution is within 40 to 45%, the temperature of the reaction tank is adjusted such that the reaction temperature of an enzyme is 55 to 60° C. The temperature of the reaction tank is adjusted, and then an enzymatic reaction is caused to occur by introducing beta-galactosidase into the reaction tank. At this time, the beta-galactosidase degrades the milk sugar to synthesize galactooligosaccharide.

An enzymatic reaction is performed for about 24 to 48 hours, and at this time, when the galactooligosaccharide content is 50 to 55 wt % or more as determined by using HPLC to analyze the sugar composition of the reaction solution, the glucose component present in the sugar solution already produced is then fermented and converted into ethanol and acetic acid by introducing yeast into the reaction tank.

At this time, as the glucose component is fermented, the contents of galactosyllactose and tetra or higher saccharides in the galactooligosaccharide are increased from about a 40 wt % level before the yeast fermentation to at least a 55 wt % level after the fermentation while the contents of the trisaccharide galactosyllactose and the tetra or higher saccharide galactooligosaccharide component in the galactooligosaccharide are relatively gradually increased, and the galactooligosaccharide content is also increased from the initial 55% level to about 75% or more. At this time, the fermentation time requires about 24 to 48 hours. When both the enzymatic reaction and the yeast reaction are completed, powdered activated carbon is introduced into the reaction tank to adsorb a coloring material of the sugar solution under stirring at 70 to 80° C. for 20 minutes to 50 minutes.

In the subsequent process, the sugar solution is filtered to remove the activated carbon and foreign matter from the sugar solution, followed by removing the acetic acid component through an ion purification process. At this time, the mechanism for the removal reaction is shown in the following Formula 1, and the produced —CH3COO(−) is removed by the ion purification process.

R—OH(−)+CH₃COO(−)/H(+)→R—CH₃COO(—)+H₂O <Formula 1>

Note 1) R: Resin body

Note2) OH(−): Functional group bonded to the resin body.

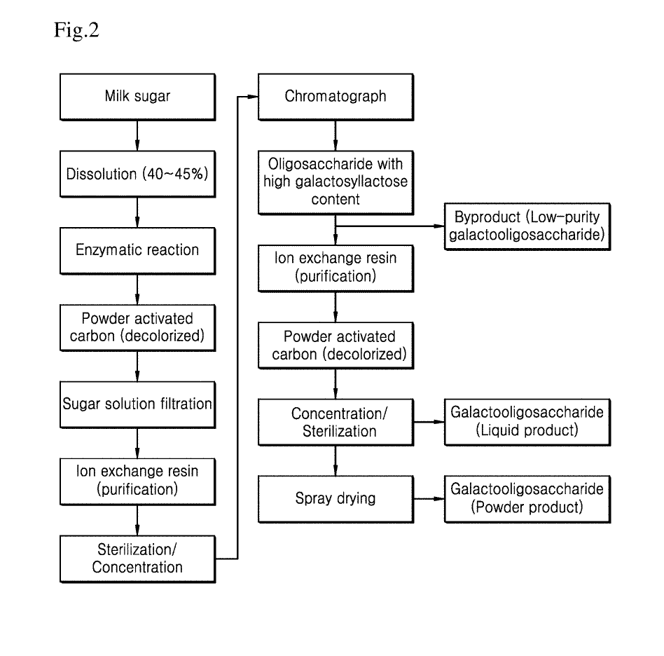

The sugar solution is subjected to ion purification to remove acetic acid, and then concentrated to a desired concentration through a concentration process. The concentration of the concentrated sugar solution may be adjusted depending on use and demand, but generally, the sugar solution is concentrated to a concentration of about 75% (FIG. 2).

Composition

The present invention relates to a method for increasing a skin water holding capacity, the method including: administering a composition including a sugar solution including galactooligosaccharide to a subject.

Further, the present invention relates to a method for suppressing water loss from the transepidermal skin of a subject, the method including: administering a composition including a sugar solution including galactooligosaccharide to the subject.

In addition, the present invention relates to a method for suppressing wrinkles and erythema in a subject, the method including: administering a composition including a sugar solution including galactooligosaccharide to the subject.

The composition may be a food composition, preferably a food composition for improving skin beauty. The composition may be a cosmetic composition, preferably a cosmetic composition for improving skin beauty. The subject is a mammal, preferably a human.

Food Composition for Improving Skin Beauty

A food composition including galactooligosaccharide according to the present invention may have an ability to improve a skin water holding capacity, an ability to suppress transdermal water loss, an ability to suppress skin wrinkles, and an ability to suppress erythema.

The food composition of the present invention may be used for skin beauty and the purpose of improving skin beauty, and may also be used for the purpose of alleviating erythema in a person suffering from erythema. The food composition may be a general food, a health supplement food, a health functional food, a functional food, and the like, but is not limited thereto, and also includes those in which the galactooligosaccharide of the present invention is added to natural foods, processed foods, general food materials, and the like. Preferably, the food composition of the present invention is a skin beauty food, a health functional food, a functional food, and the like.

Cosmetic Composition for Improving Skin Beauty

When the composition of the present invention is formulated into a cosmetic, the composition includes components typically used in a cosmetic composition in addition to galactooligosaccharide as a component included in a cosmetic composition of the present invention, and may include an auxiliary used in a typical cosmetic composition such as, for example, a stabilizer, a solubilizer, a pigment, vitamins, and an antioxidant.

The benefits and features of the present invention, and the methods of achieving the benefits and features will become apparent with reference to exemplary embodiments to be described below in detail along with the accompanying drawings. However, the present invention is not limited to the exemplary embodiments to be disclosed below, but may be implemented in various other forms, and the present exemplary embodiments are provided for rendering the disclosure of the present invention complete and for fully representing the scope of the invention to a person with ordinary skill in the technical field to which the present invention pertains, and the present invention will be defined only by the scope of the claims.

<Materials and Methods>

As milk sugar and enzymes which are raw materials, commercially available products were purchased and used.

HPLC Analysis of Sugar Composition in Sugar Solution

Ultrapure water (specific conductivity value: 0.05 uS/cm or less) was added to a sample sugar solution, and the sugar solution was diluted so as to have a sample sugar solution concentration of about 2.5 to 3.0%. (A refractometer was used for the analysis of a sample concentration). After the concentration of the sample sugar solution was adjusted, foreign matter contained in the sample solution was removed by using a 0.2 to 0.45 um filter, and then the sample was used as a sample for HPLC analysis.

An analysis of galactooligosaccharide content (DB%) was measured by using HPLC. As a column for HPLC analysis, a polyamine-based column was used, the contents of glucose, galactose, and lactose in the galactooligosaccharide were obtained, and the galactooligosaccharide content was obtained in accordance with the following Formula 2.

Galactooligosaccharide content(DB %)=100−(glucose content+galactose content+milk sugar content) <Formula 2>

Note 3) DB %: Dry Basis. Refers to being based on anhydrides.

### EXAMPLE 1

### Preparation of Sugar Solution with High Galactooligosaccharide Content

After lactose (milk sugar) was introduced into a reaction tank, hot water was added thereto, and the concentration of the milk sugar solution was adjusted to about 43% by operating a stirrer. When the concentration of the milk sugar solution was within 40 to 45%, the temperature of the reaction tank was adjusted such that the reaction temperature of an enzyme was 58° C. The temperature of the reaction tank was adjusted, and then an enzymatic reaction was caused to occur by introducing beta-galactosidase into the reaction tank. At this time, the beta-galactosidase degraded the milk sugar to synthesize galactooligosaccharide.

An enzymatic reaction was performed for about 36 hours, and at this time, when the galactooligosaccharide content was 53 wt % or more as determined by using HPLC to analyze the sugar composition of the reaction solution, the glucose component present in the sugar solution already produced was then fermented and converted into ethanol and acetic acid by introducing yeast into the reaction tank.

At this time, as the glucose component was fermented, the contents of galactosyllactose and tetra or higher saccharides in the galactooligosaccharide were increased from about a 40wt % level before the yeast fermentation to at least a 55 wt % level after the fermentation while the contents of the trisaccharide galactosyllactose and the tetra or higher saccharide galactooligosaccharide component in the galactooligosaccharide were relatively gradually increased, and the galactooligosaccharide content was also increased from the initial 55% level to about 75% or more. At this time, the fermentation time required about 40 hours. When both the enzymatic reaction and the yeast reaction were completed, powdered activated carbon was introduced into the reaction tank to adsorb a coloring material of the sugar solution under stirring at 75° C. for 40 minutes.

In the subsequent process, the sugar solution was filtered to remove the activated carbon and foreign matter from the sugar solution, followed by removing the acetic acid component through the ion purification process in Formula 1.

The sugar solution was subjected to ion purification to remove acetic acid, and then concentrated through a concentration process.

### COMPARATIVE EXAMPLE 1

A sugar solution was prepared in the same manner as in Example 1, except that the yeast reaction was omitted. The method for preparing the sugar solution is as follows.

After lactose (milk sugar) was introduced into a reaction tank, hot water was added thereto, and the concentration of the milk sugar solution was adjusted to about 43% by operating a stirrer. When the concentration of the milk sugar solution was within 40 to 45%, the temperature of the reaction tank was adjusted such that the reaction temperature of an enzyme was 58° C. The temperature of the reaction tank was adjusted, and then an enzymatic reaction was caused to occur by introducing beta-galactosidase into the reaction tank. At this time, the beta-galactosidase degraded the milk sugar to synthesize galactooligosaccharide.

The enzymatic reaction was performed for about 36 hours, and after the enzymatic reaction was completed, powdered activated carbon was introduced into the reaction tank to adsorb a coloring material of the sugar solution under stirring at 75° C. for 40 minutes.

In the subsequent process, the sugar solution was filtered to remove the activated carbon and foreign matter from the sugar solution, followed by removing the acetic acid component through the ion purification process in Formula 1. Next, the sugar solution was subjected to ion purification to remove acetic acid, and then concentrated through a concentration process.

### COMPARATIVE EXAMPLES 2 to 4

Three types of commercially available sugar solutions were purchased and used in Comparative Examples 2 to 4, respectively.

### EXPERIMENTAL EXAMPLE 1

### Analysis of Composition of Sugar Solution

For the sugar solution in Example 1 and Comparative Examples 1 to 4, the sugar compositions were analyzed by using HPLC.

As a result, it could be seen that the contents of the trisaccharide galactosyllactose of galactooligosaccharide and tetra or higher saccharide galactooligosaccharide in the sugar solution in Example 1 were significantly higher than those in Comparative Examples 1 to 4 (Table 1).

TABLE 1

| Sugar composition | | Comparative | Comparative | Comparative | Comparative |
|---|---|---|---|---|---|
| (Sugar profile) | Example 1 | Example 1 | Example 2 | Example 3 | Example 4 |
| Glucose + Galactose | 5.0 | 19.5 | 17.6 | 20.1 | 20.0 |
| Galactobiose + Allolactose | 19.7 | 22.3 | 21.4 | 20.6 | 17.5 |
| Milk Sugar (Lactose) | 14.7 | 21.8 | 22.5 | 8.6 | 21.5 |
| Galactosyllactose | 40.8 | 23.3 | 20.4 | 23.5 | 22.8 |
| Tetra or higher saccharides | 15.2 | 13.1 | 18.1 | 17.2 | 18.2 |
| of galactooligosaccharide | | | | | |
| *Total | 75.7 | 58.7 | 59.9 | 61.3 | 58.5 |
| Galactooligosaccharides | | | | | |

Unit: DB %:
DB %: Based on anhydrides (Dry Basis, wt %)
*Total galactooligosaccharides
:Galactobiose + Allolactose + Galactosyllactose + Tetra or higher saccharides of galactooligosaccharide

### EXPERIMENTAL EXAMPLE 2

### Comparative Experiment of Improving Skin Beauty in Animal Model

For the sugar solutions in Example 1 and Comparative Examples 1 to 4, the ability to improve skin beauty was evaluated. Skin beauty improvement ability tests were compared with one another through skin water holding capacity, transepidermal water loss and erythema suppression ability experiments.

For the animal model, 36 hairless male SKH-1 mice (5 week old) in total were purchased and divided into 6 groups of 6. Specifically, the mice were bred in a plastic cage under conditions maintained at 24±1° C. and 60% RH humidity, and allowed to differentiate day and night at intervals of 12 hours, and in the case of diet, the mice were subjected to experimental accommodation (adaptation) time by applying water and a general diet.

After the adaptation time was completed, a control was treated with Ultraviolet B (UVB) while ingesting a general diet for 12 weeks. Meanwhile, an experimental group was treated with UVB while being fed the general diet and an additional diet of 100 mg of a sample (sugar solution) per 1 kg of mouse. The test measurement was performed immediately after completion of the adaptation time and 12 weeks after completion of the test.

At this time, the ultraviolet B (UVB) treatment was performed in accordance with the method described in the paper (Gueniche A, Hennino A, Goujon C et al. (2006) Improvement of atopic dermatitis skin symptoms by Vitreoscilla filiformis bacterial extract. Eur J Dermatol 16:380-4).

Furthermore, as a ultraviolet B (UVB) treatment apparatus, FLB2OSBL (Sankyo Denki, Japan) was used. The back of the experimental animal was irradiated and exposed to UV rays, and UV rays with 270 to 400 nm (output peak at 313 nm) as a wavelength range of a UV light source for exposure were used.

<2-1> Evaluation of Skin Water Holding Capacity

The skin water holding capacity was measured by using Corneometer CM825 (Courage+Khazaka electronic GmbH, Cologne, Germany). The corneometer is an apparatus using a principle of measuring the capacitance of current conducting through a conduction gap in contact with the surface of the skin, and since the skin water holding capacity of the skin and the capacitance are proportional to each other, the drier the skin is, the lower the value is, and the measurement unit is an arbitrary unit (AU) which is a relative value to skin humidity. As a measurement value of the skin water holding capacity, a value appearing by closely attaching a sensor to the surface of the skin of a site to be measured, and then lightly pressing the sensor to the surface was used. Statistical analysis was performed by using SPSS 12.0 (SPSS Inc., Chicago, Ill.).

As a result, all of the sugar solutions in Example 1 and Comparative Examples 1 to 4 had excellent skin water holding capacities compared to the control. In particular, the sugar solution in Example 1 was excellent in water holding capacity compared to Comparative Examples 2 to 4. The sugar solutions in Comparative Examples 1 to 4 had a combined content of the trisaccharide galactosyllactose and tetra or higher saccharides of galactooligosaccharide in the sugar composition in a range from 36 to 41%, whereas the sugar solution in Example 1 had a content of the trisaccharide galactosyllactose and tetra or higher saccharides of galactooligosaccharide in the sugar composition of approximately 55%. Therefore, it was determined that the higher the combined content of galactosyllactose and tetra or higher saccharides of galactooligosaccharide was, the higher the skin water holding capacity was (Table 2).

TABLE 2

| | Skin water |
|---|---|
| | holding capacity |
| | (average, %) |
| Control | 44.3 |
| Example 1 | 56.3 |
| Comparative Example 1 | 48 |
| Comparative Example 2 | 46 |
| Comparative Example 3 | 51 |
| Comparative Example 4 | 50 |

<2-2> Evaluation of Transepidermal Water Loss

The transepidermal water loss (TEWL) was measured by using Tewameter TM300 (Courage+Khazaka electronic GmbH, Cologne, Germany). For the transepidermal water loss (TEWL), the amount of moisture evaporating from the epidermis is calculated by obtaining vapor pressure due to moisture diffusing from the surface of the skin into the air by Fick's law. For the evaporation of moisture through the stratum corneum, an evaporation amount (g/h/m²) can be obtained within 30 to 50 seconds from the physiology of the epidermis, which dynamically changes during a dry state of the skin due to passive diffusion (Vertuani et al., 2003). For the evaluation, a measurement was performed for about 20 seconds until the value was stabilized, and an average of the three values except for the maximum value and the minimum value among the 5 stabilized values was used. The worse the dry symptoms were, the higher the measured value was, and the measurement coefficient was recorded as g/h/m².

As a result, it was confirmed that all of the sugar solutions in Example 1 and Comparative Examples 1 to 4 suppressed the transepidermal water loss (%) compared to the control.

In particular, it was confirmed that the sugar solution in Example 1 had a higher transepidermal water loss suppression than those of the commercially available sugar solutions in Comparative Examples 2 to 4.

The sugar solutions in Comparative Examples 1 to 4 had a combined content of the trisaccharide galactosyllactose and tetra or higher saccharides of galactooligosaccharide in the sugar composition in a range of 36 to 41%, whereas the sugar solution in Example 1 had a content of the trisaccharide galactosyllactose and tetra or higher saccharides of galactooligosaccharide of approximately 55%, so that it was determined that the higher the combined content of galactosyllactose and tetra or higher saccharides of galactooligosaccharide was, the lower the transepidermal water loss was (Table 3).

TABLE 3

| | Transdermal |
|---|---|
| | water loss |
| | (average, %) |
| Control | 12 |
| Example 1 | 7.4 |
| Comparative Example 1 | 8.6 |
| Comparative Example 2 | 8.8 |
| Comparative Example 3 | 8.5 |
| Comparative Example 4 | 8.7 |

<2-3> Evaluation of Erythema Index

The erythema index (erythema level) was measured by using Mexameter MX18 (Courage+Khazaka electronic GmbH, cologne, Germany) The measurement result is represented by melanin index (MI) and erythema index (EI) within 1 second after a sensor is brought into contact with the skin (Christian, 2003). The worse the erythema symptoms are, the higher the measured value is.

As a result, it could be confirmed that all of the sugar solutions in Example 1 and Comparative Examples 1 to 4 suppressed erythema compared to the control. In particular, Example 1 had an excellent erythema suppression ability.

The sugar solutions in Comparative Examples 1 to 4 had a combined content of the trisaccharide galactosyllactose and tetra or higher saccharides of galactooligosaccharide in the sugar composition in a range of 36 to 41%, whereas the sugar solution in Example 1 had a content of the trisaccharide galactosyllactose and tetra or higher saccharides of galactooligosaccharide of approximately 55%, so that it was determined that the higher the combined content of galactosyllactose and tetra or higher saccharides of galactooligosaccharide was, the lower the erythema index was (Table 4).

TABLE 4

| | Measurement |
|---|---|
| | result of |
| | erythema index |
| Control | 320 |
| Example 1 | 250 |
| Comparative Example 1 | 282 |
| Comparative Example 2 | 278 |
| Comparative Example 3 | 281 |
| Comparative Example 4 | 280 |

<2-4> Evaluation of MMP Suppression Ability

A TRIzol reagent (Invitrogen, Carlsbad, Calif.) was purchased, and total RNA was separated from a skin sample of an animal tested in accordance with the manufacturer's protocol. Complementary DNA (cDNA) was synthesized by reverse-transcribing the RNA of 1 mg of the skin sample using a REvertAid First-Strand DNA synthesis kit (Thermo Scientific Fisher, Waltham, Mass.). The synthesized sample was treated with RQ1 RNase-free Dnase I (Promega, Madison, Wis.) in accordance with the manufacturer's protocol. The synthesized cDNA was applied to a real-time polymerase chain reaction (real-time PCR by using a Power Taqman Master Mix kit (Applied Biosystems, Foster City, Calif.). For a primer and a probe used in the present experiment, MMP2 (GenBank ID: NM_008610.2) and MMP9 (GeneBank ID: NM_008084.2) were used as an internal standard material.

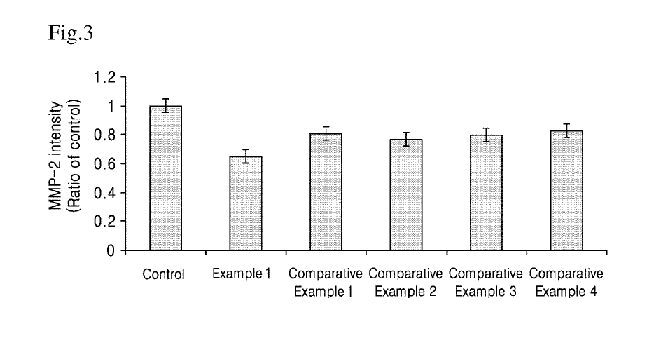

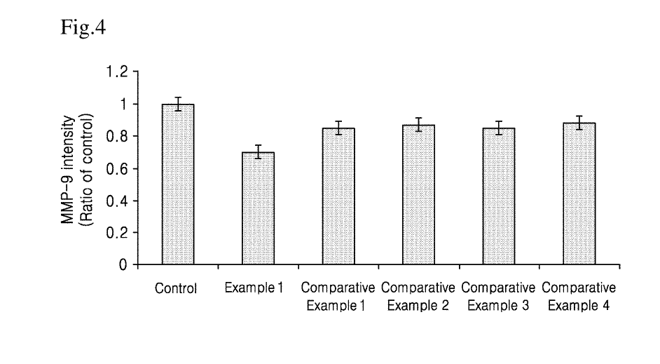

An experimental result of suppression effects of MMP-2 and MMP-9 in an animal model determined by the measurement as described above is shown in following FIGS. 3 and 4, respectively.

As a result, all of the sugar solutions in Example 1 and Comparative Examples 1 to 4 had effects of suppressing MMP-2 and MMP-9 compared to the control. In particular, it was confirmed that in Example 1, MMP-2 and MMP-9 were more significantly suppressed than in Comparative Examples 2 to 4 which are commercially available sugar solutions. This result indicates that galactooligosaccharide in a sugar solution has a mechanism for alleviating wrinkles, improving a skin water holding capacity, decreasing transepidermal water loss, and the like by suppressing MMP-2 and MMP-9.

Further, it was determined that the MMP-2 suppression effect and MMP-9 suppression effect of the sugar solution in Example 1 were effects occurring because the combined content of the trisaccharide galactosyllactose of galactooligosaccharide and tetra or higher saccharides of galactooligosaccharide in the sugar solution was higher than those of the sugar solutions in Comparative Examples 1 to 4 by about 20% or more (FIGS. 3 and 4).

### EXPERIMENTAL EXAMPLE 3

### Evaluation of Skin Condition Improving Ability

A clinical evaluation was performed by using the sugar solution in Example 1, which was confirmed to be excellent in skin beauty effects in Experimental Examples 1 and 2. At this time, skin-related parameters were examined by allowing a healthy adult to ingest a sample for 12 weeks. The test method and the design were tested through a stratified block randomized allocation of a control and an experimental group (group administered with the sugar solution in Example 1) using double blinded, randomized, and placebo controlled methods. The experimental group was fed 1.0 g of the sample twice a day, and the control was fed a simple placebo.

Statistical analysis of the clinical test result was performed by using SPSS 12.0 (SPSS Inc., Chicago, Ill.). Significant differences between the control and the experimental group were compared with one another by using a Student's t-test. Each experimental value was represented as mean±SEM (standard deviation) for a measurement item.

<3-1> Evaluation of Skin Water Holding Capacity

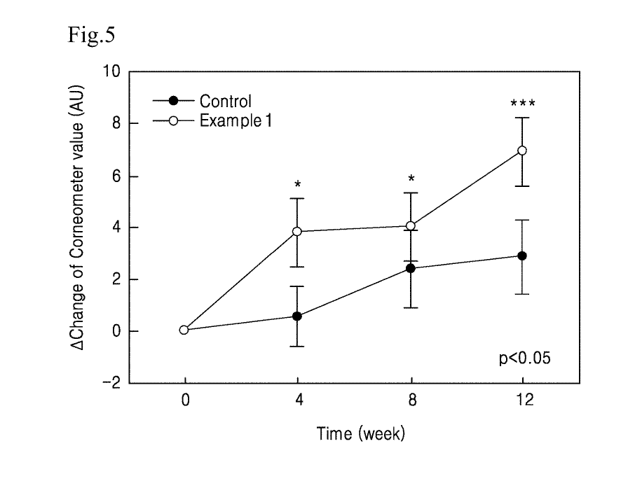

As a result of measuring the skin water holding capacity, the variation in skin water in the experimental group which had ingested the sugar solution in Example 1 was determined to be statistically significantly higher than that of the control. It was confirmed that as time passed, the skin water holding capacity of the experimental group was increased more than that of the control. It was determined that the increase in skin water holding capacity resulted from a mechanism in which galactooligosaccharide in the sugar solution significantly suppresses MMP-2 and MMP-9 to improve a skin water holding capacity as confirmed in Experimental Example 2 (FIG. 5).

<3-2>Evaluation of Transepidermal Water Loss

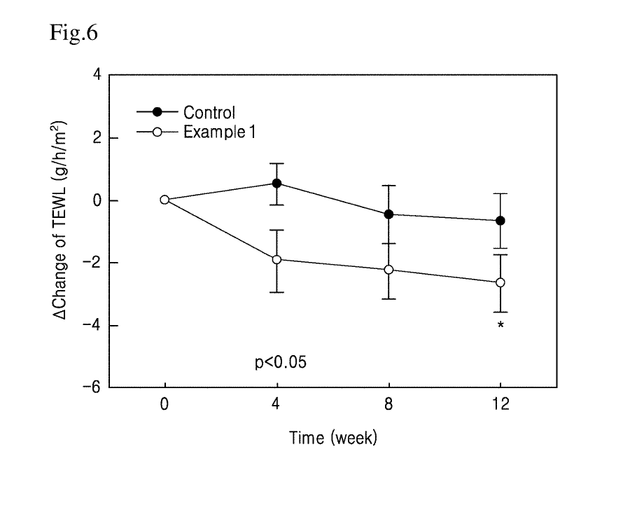

As a result of measuring a change in transepidermal water loss measured for 12 weeks, a group which had ingested the sugar solution in Example 1 was statistically significantly suppressed compared to the control. It was confirmed that as time passed, the transepidermal water loss in the skin of the experimental group was significantly suppressed compared to that of the control, coinciding with the result in Experimental Example 2 (FIG. 6).

<3-3> Evaluation of Skin Wrinkle Alleviating Ability

The skin wrinkle alleviating ability of the sugar solution in Example 1 was evaluated by manufacturing a simulation plate and analyzing the simulation plate. Before a sample was ingested and 12 weeks after the sample was ingested, 11 mm (diameter) adhesive paper was attached to the skin, and then semi-transparent silicone was mixed in a small plastic cup (Courage+Khazaka electronic GmbH) containing a base material and a catalyst. The layer of the silicone mixture was spread on a limited region of the adhesive paper and dried for 5 minutes. When the silicone mixture was sufficiently dried, the manufactured simulation plate was stored in a tracing paper envelope until the analysis was completed. Wrinkles were analyzed by using skin-visiometer SV 600 (Courage+Khazaka electronic GmbH). A total wrinkle area (mm²), a percentage of wrinkle area (%), an average of wrinkle depth (um), the number of wrinkles, and the like were measured. At this time, no skin care product was used on a measured site for at least two hours before the measurement.

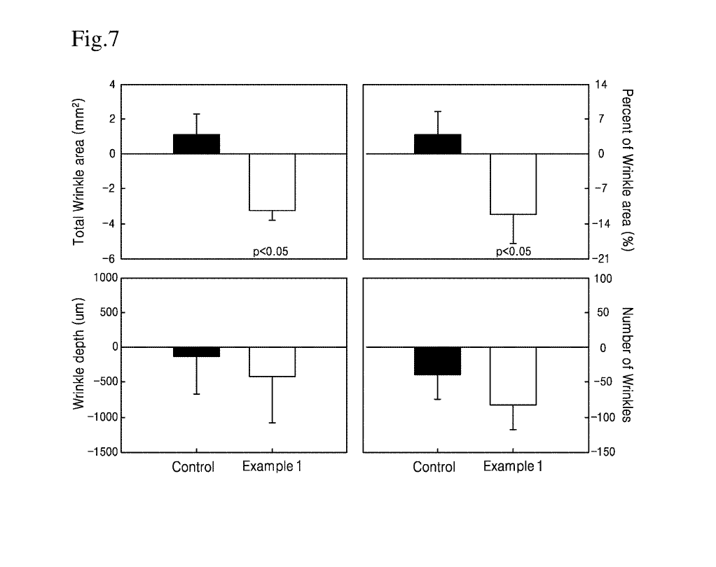

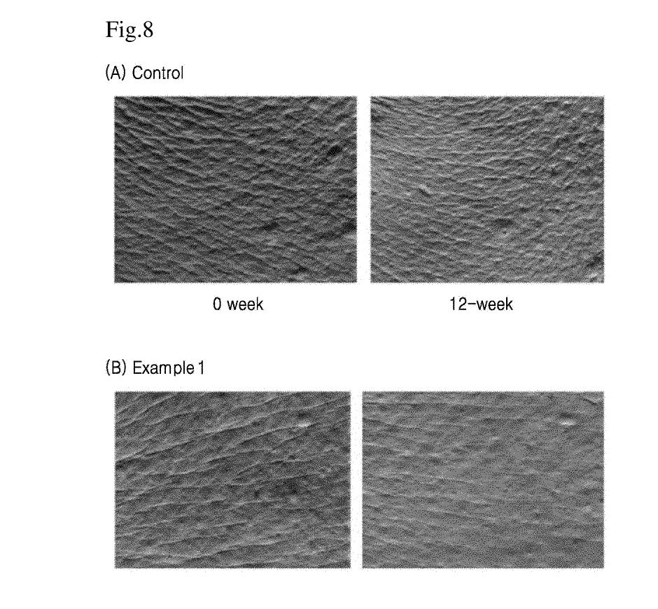

As a result, it was confirmed that in a group treated with the sugar solution in Example 1, the total wrinkle area and the wrinkle depth were significantly decreased 12 weeks later compared to those in the control (FIG. 7), and it was also confirmed that the group treated with the sugar solution in Example 1 had a lower number of wrinkles after completion of the test than that at the start of the test (FIG. 8).

The food composition and cosmetic composition of the present invention have effects of increasing a skin water holding capacity, inhibiting transepidermal water loss, alleviating skin wrinkles, and alleviating erythema.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. A method for increasing a skin water holding capacity, the method comprising:
administering a composition comprising a sugar solution comprising galactooligosaccharide to a subject in need thereof,
wherein the sugar solution comprises galactobiose and allolactose, and an amount of galactobiose and allolactose among sugars in the sugar solution ranges from 15 wt % to 22 wt % on dry basis, and
wherein the method suppresses wrinkles and erythema in the subject in need thereof.

2. The method of claim 1, wherein the sugar solution further comprises galactosyllactose.

3. (canceled)

4. The method of claim 1, wherein the sugar solution further comprises lactose, and an amount of lactose among sugars in the sugar solution ranges from 5 wt % to 18 wt % on dry basis.

5. The method of claim 2, wherein an amount of galactosyllactose among sugars in the sugar solution ranges from 35 wt % to 50 wt % on dry basis.

6. The method of claim 2, wherein among sugars in the sugar solution, an amount of galactosyllactose is larger than a combined amount of galactobiose and allolactose.

7. The method of claim 1, wherein the sugar solution comprises glucose and galactose, and an amount of glucose and galactose among sugars in the sugar solution ranges from 3 wt % to 8 wt % on dry basis.

8. The method of claim 1, wherein the method suppresses water loss from the transepidermal skin of the subject.

9. (canceled)

10. The method of claim 1, wherein the composition is a food composition.

11. The method of claim 1, wherein the composition is a cosmetic composition.
